(19) United States
(12) Patent Application Publication
(10) Pub. No.: US 2016/0030230 A1
(43) Pub. Date: Feb. 4, 2016

(54) DEVICE FOR THE HINDRANCE OF SNORING AND OBSTRUCTIVE SLEEP APNEA

(71) Applicant: NiteCap, Inc., Simpsonville, KY (US)

(72) Inventor: Thomas R. Barito, Simpsonville, KY (US)

(21) Appl. No.: 14/881,235

(22) Filed: Oct. 13, 2015

Related U.S. Application Data
(63) Continuation-in-part of application No. 14/164,088, filed on Jan. 24, 2014.
(60) Provisional application No. 61/849,317, filed on Jan. 25, 2013.

(51) Int. Cl. A61F 5/56

(52) U.S. Cl. CPC A61F 5/56

(57) ABSTRACT

A head-wearable device is disclosed. The head-wearable device includes a headgear support structure, a band structure for securing the headgear support structure to the wearer's head, and a protrusion assembly affixed to the headgear superstructure for hindering the wearer's head from assuming a supine position during sleep. A method of sleeping is also disclosed.

### RELATED APPLICATION

This application is a continuation-in-part of U.S. patent application Ser. No. 14/164,088, filed Jan. 24, 2014, which claims the benefit of U.S. Provisional Application No. 61/849,317 filed on Jan. 25, 2013.

### BACKGROUND

The term snoring generally refers to a rough or hoarse sound that arises from a person's mouth while sleeping. The problems caused by snoring are both social and medical, affecting those who sleep with or near the person snoring and sometimes signaling a more serious problem known as obstructive sleep apnea. It is well known that sleeping in the supine position (on the back) promotes airway obstruction and the resultant snoring or obstructive sleep apnea. Depending on the degree of blockage, there can be simple snoring or a momentary, total blockage of the airflow, known as obstructive sleep apnea. Obstructive sleep apnea is a potentially very serious condition. The oxygen starvation it induces can cause the person to partially awaken in order that muscle tension can open the airway and get air into the lungs. During these episodes, the heart must pump faster to keep up with the oxygen demand of the body. This condition can result in excessive daytime sleepiness, irregular heartbeats, and after many years of suffering this condition, may lead to elevated blood pressure and heart enlargement. Persons with obstructive sleep apnea may spend little of their sleeping hours in the deep sleep stages, such as REM sleep, that are essential for good health.

Many inventions aimed at controlling snoring and obstructive sleep apnea have been proposed to deter supine sleeping. Several of these inventions employ methods to restrict torso movement so that the patient avoids sleeping in the supine position (on the back). Others utilize pillows to support the torso and to block movement to the supine position. Still other inventions involve objects attached to the back of garments that extend a distance behind the device and are intended to provide some degree of discomfort when the wearer or patient attempts to sleep in the supine position. The most well-known version of this approach is the configuration in which tennis balls are sewn into the back of a shirt.

The problem with these devices is that they are aimed at controlling torso movement and do not deter the patient from sleeping with the head in a supine position. It is very likely that while the torso may be stopped from sleeping in the supine position, the head will rotate independently to a supine position. The problem with this head position is that a primary cause for obstructive sleep apnea is in the head and not the torso. In deep sleep, the relaxation of the tongue and lower jaw combined with supine sleeping result in the narrowing of the breathing passage for patients with obstructive sleep apnea. The end result is obstructed breathing that can result in the complications outlined previously.

Examples of headgear for mitigation of snoring and obstructive sleep apnea include U.S. Pat. No. 5,687,743 issued to Goodwin, directed to a head strap assembly having chin straps to hold the wearer's mouth closed during sleep. However, this device does not restrict the wearer's head from assuming a supine position. Simply keeping the mouth closed to force nasal breathing is not adequate to mitigate or stymie obstructive sleep apnea, or snoring in general, as the tongue will still fall back onto the soft palate and block the breathing passages, including the nasal openings into the throat, when the head is in a supine position. Snoring and obstructive sleep apnea still can occur with the mouth closed while sleeping on one's back.

U.S. Pat. No. 8,118,030 issued to Bugeja teaches a headband with a cushion attached to the headband, so that the wearer may wear the headband with the cushion covering the back of the head, in the hopes that the cushion would restrict the head from assuming a supine position. However, this device is very prone to rotation and sliding off the wearer's head during sleep. In addition, the cushion may not be obtrusive enough to prevent some wearers from resting the head on the cushion in a supine position, especially on a soft pillow. Other devices include U.S. Patent Application Publication No. 2008/0120765 to Zhu, wherein a cap to be worn on the head is taught, having a chinstrap assembly attached to the cap. While the chinstrap may function to support the lower jaw and prevent the mouth from opening during sleep, secured to the head by the cap, the device does not impede the wearer from turning the head or body to a supine position. Along a similar vein, Japanese Patent Nos. JP 2002-272771 A and JP 2003-164480 A, issued to Koyanagi and Koga, respectively, both disclose a cap to be worn during sleep, and having a chinstrap to hold the mouth closed, forcing breathing through the nose. Again, these devices do not prevent or otherwise discourage the wearer from sleeping with the head in a supine position. Thus, there is a need for a simple device that effectively impedes the head from assuming a supine position during sleep, while at the same time providing a secure and comfortable fit to the head.

### SUMMARY

This disclosure addresses the issues raised in the preceding section, and thus may mitigate or curb snoring and obstructive sleep apnea for many individuals afflicted with these conditions more effectively than the proposed solutions cited in the examples above. The wearable headgear described herein includes, generally, i) a headgear structure at least partially encircling the cranium when worn on the head by the wearer for supporting the headgear device on the head; ii) a network of bands or straps, wherein at least one band or strap at least partially encircles the head of a wearer around the face, and passes under the wearer's chin for securing the inventive headgear device to the head of the wearer, and to hold the wearer's mouth closed when sleeping; iii) one or more rigid or semi-rigid extensions protruding from the rear portion of headgear for hindering the wearer's head from assuming a supine position during sleep.

The preferred sleep position may thus be the lateral recumbent or oblique body positions, where the wearer sleeps with the body resting primarily on its side, or with the head turned to the side. The headgear structure provides a means to secure the extensions to the wearer's head. As stated above, the headgear device comprises a facial band or strap that is worn around the chin to provide tensile forces around the lower jaw to hold the mouth substantially closed while the wearer sleeps, forcing the wearer to breathe through the nose. This band or strap, referred to in this disclosure as the restraining chinstrap, extends along both sides of the face, on either side of the sagittal plane dividing the head, extending from both sides of the chin to the headgear structure to which it may be integrally attached. In one embodiment, the chinstrap may also bifurcate into two portions that pass both directly in front and behind the wearer's ears and in close proximity thereto, in order to more tenaciously secure and stabilize the headgear on the wearer's head so that it does not rotate or come off during sleep, using the wearer's ears as anchoring structures.

The chinstrap may be formed from elastic or non-elastic materials, and be formed as a contiguous loop extending from the headgear structure on both sides of the face, or it may comprise two elastic or non-elastic segments that may be fastened together by the wearer when putting on the inventive headgear. In both forms, the chinstrap may have an adjustable tension. For embodiments with attachable segments, adjustable fastening means are disposed on the free ends of the segments. In all embodiments, the chinstrap is permanently affixed to the headgear structure and may be integral therewith. In this disclosure, the combined headgear structure and chinstrap is referred to as a headgear superstructure. Reiterating, the chinstrap may be a simple strap or band, or may comprise several bands or straps formed in a network.

Furthermore, it preferably serves a dual functionality, i) to secure the headgear structure to the wearer's head, and stabilize it such that it may not rotate significantly during sleep, and ii) to provide sufficient tensile force so that the wearer's mouth remains substantially closed during sleep, forcing the wearer to breathe through the nose.

In one embodiment, the headgear structure may be substantially in the form of a cap covering the cranium of the wearer, wherein a restraining chinstrap is attached to the perimeter of the cap. The cap may be worn in close contact with the wearer's head so that the body of the cap distributes any tensile forces evenly over the wearer's cranium, making for a comfortable fit. A means may be provided to adjust the tightness of the cap on the wearer's head, such as an adjustable strap disposed along the perimeter of the cap, as is commonly found in baseball-style caps. In other embodiments, the cap may comprise an elastic band or non-elastic adjustable band disposed along the perimeter that may fit snugly around the wearer's head when adjusted by the wearer to secure the cap.

In all embodiments of the inventive headgear, special rigid structures protrude behind the headgear superstructure. The rearwardly protruding structures preferably have a horizontal aspect of sufficient width and depth to hinder the wearer's head from assuming a supine position. In some embodiments, the protruding structures are formed into an assembly of one or more rigid plate-like or flap-like structures extending rearwardly from the occipital portion of the headgear superstructure. The plane of the plate-like or flap-like structures may be oriented substantially horizontally, having width and depth of sufficient extent to be a hindrance to head rotation from a lateral position of the wearer's head to a supine position. Alternatively, the plate-like or flap-like structures may be oriented vertically.

An assembly of two or more plate members may include a strut-like structure between the plates to maintain a separation between them. An example of an embodiment using such an assembly is a protrusion assembly having two horizontal plate or flap members extending divergently to the rear from a substantially common point of attachment, forming an acute angle between them. The diverging plates or flaps create a third dimensional aspect in the protrusion assembly by providing a vertical extent of the rear protrusion. This third-dimensional aspect provides a bulkiness to the protrusion assembly, retaining its ability to provide a hindrance to head rotation to a supine position during sleep if inadvertently folded against the head, or tilted in an upward or downward orientation. To this end, the protrusions may have a wedge shape or other three-dimensional shape to prevent acute folding or tilting of the protrusion assembly. In all embodiments of the inventive headgear, protrusions may be affixed to the headgear superstructure (combined headgear structure and facial band or strap) forming a joint that is integral with the headgear superstructure, such that the joint may resist bending or folding by the weight of the wearer's head, or by forces that may be reasonably exerted by the wearer's neck muscles to rotate or tilt the head. Several embodiments of the disclosed device are discussed in detail below. It will be understood by persons skilled in the art that many other embodiments of the invention are possible, based on the broadest description of this invention provided above. For example, the protrusion assembly may be configured in several forms that accomplish the objective of impeding or hindering the patient from sleeping with the head assuming a supine orientation.

These and other features may be best understood from the following drawings and specification.

### BRIEF DESCRIPTION OF THE DRAWINGS

FIG. 1a shows top and side views of the preferred embodiment of headgear, where the headgear support structure is a cap.

FIG. 1b is a side view of the headgear, showing the members of the protrusion assembly extending rearward from the cap, and having an example of a supporting structural member, in this case a web, inserted between the two bill-shaped plates to maintain the separation angle between the plates.

FIG. 1c is a top view of an alternative embodiment of the headgear, showing a tapered protrusion member.

FIG. 1d is a three-dimensional view of the headgear, showing an alternative embodiment of the protrusion assembly having a single horizontal bill-shaped member, and a vertical crest member to prevent the horizontal member from folding upwards.

FIG. 1e is an oblique view of an alternative embodiment of the headgear, showing a ball-shaped protrusion member extending from a cap.

FIG. 1f is an oblique view of an alternative embodiment, showing a single unit wedge-shaped protrusion assembly member extending from a cap.

FIG. 1g is a top view of the protrusion assembly showing radius.

FIG. 1h is a top view of the protrusion assembly showing dimensions.

FIG. 2a is a side view of the headgear worn on the head, showing a simple chinstrap.

FIG. 2b is a side view of the headgear worn on the head, showing a simple bifurcated chinstrap.

FIG. 2c. is a side view of the headgear worn on the head, showing a bifurcated chinstrap having widened straps closely surrounding the ear, providing a more secure fit by using the ear as an anchor structure.

FIG. 3 is a side view of the headgear without a cap, having a chinstrap with two securing straps running diagonally towards the rear of the head.

FIG. 4 shows a subject asleep wearing the headgear.

FIG. 5 is an oblique view of the headgear with a cap worn by a wearer.

FIG. 6 is a back view of the cap worn by a wearer.

FIG. 7 is an oblique view of the protrusion assembly without a cap.

### DETAILED DESCRIPTION

### Definitions of Terms

By the term “bill-shaped structure,” it is meant a plate-like or a flap-like structure having a shape, size and aspect ratio similar to the bill of a cap such as a baseball-style cap or a visor worn on the head. It will be understood that the general shape of such a structure may be characterized by having a large horizontal to vertical aspect ratio, and extending substantially horizontally from the headgear superstructure when worn on the upright head. The term “bill-shaped” may imply forms ranging from substantially rectilinear forms to substantially round forms, where the round forms may have circular to ellipsoidal to parabolic outlines. It will be understood that no particular form or shape is implied or to be construed by this term. Moreover, more complex shapes such as scalloped, ridged, or curved embodiments of the plate-like or flap-like structures are also included in this definition.

By the term “rigid,” it is meant that the structures are sufficiently stiff to resist acute bending by the weight of the wearer's head, or by forces exerted on the structure by the neck muscles of the wearer when unconsciously attempting to roll the head to a supine position during sleep.

By the term “web,” it is meant a vertical rigid structure between two or more bill-shaped plate members of the protrusion assembly, as the web separating the flanges of an I-beam, intended to withstand compressive forces and maintain separation between the multiple members of the protrusion assembly.

By the term “acute,” it is meant an acute angle temporarily or permanently induced in a structure by the bending or folding a portion of a structure where the angle did not exist in the structure before the bending or folding action. In this context, the structure may be a headgear superstructure and protrusions extending therefrom.

The term “headgear support structure” is meant to refer to the portion of the headgear that at least partially encircles the cranium of the wearer. In most embodiments, this portion of the headgear is its primary structural member, acting as an anchor affixed to the top or sides of the wearer's head.

The terms “protrusion,” “protrusion structure,” “protrusion structure assembly” and “protrusion assembly,” refer to rearward extensions emanating from the headgear support structure.

The terms “band,” “band network,” “strap” and “chinstrap” are related terms to distinguish bands or straps intended to secure the headgear support structure to the head of the wearer. The term “band” is generally directed to an elongated structure for securing the headgear support structure to the head, and may be elastic or not, whereas “strap” is generally directed to a non-elastic elongated flexible structure.

The term “headgear superstructure” is meant to be construed as a the combined structure comprising the headgear support structure and the band structure in this disclosure, and to be used preemptively to cover permutations of the embodiments of the inventive headgear, wherein protrusion structures may be affixed to points anywhere on the superstructure. For example, protrusion structure assemblies may be affixed to the chinstrap (band that at least partially encircles the wearer's head along the cranial-caudal axis) instead of the headgear support structure itself.

By the term “cranium,” the usual anatomical meaning is assumed, viz., the top portion of the wearer's head, or top of the head.

By the term “occipital,” the usual anatomical meaning is assumed, viz., the rear portion of the wearer's head, or back of the head.

The term “cranial-caudal axis” is defined in the context of human anatomy. For the purposes of this disclosure, it is intended to be construed as a vertical axis of the head running from top of the cranium to the base of the skull, and used herein for purposes of defining relative direction and spatial orientation with respect to the wearer's body.

The term “rostral-occipital axis” is defined in the context of human anatomy. For the purposes of this disclosure, it is intended to be construed as a horizontal axis of the head running from the face, or rostrum, to the back of the head, or occipital region, and used herein for purposes of defining relative direction and spatial orientation with respect to the wearer's body.

The term “sagittal” is to be construed in the standard definition, referring to a vertical plane dividing the human body along the axis of bilateral symmetry, or left and right sides, and used herein for purposes of defining relative direction and spatial orientation with respect to the wearer's body.

The term “transverse” is to be construed in the standard definition, referring to a horizontal plane dividing the body into an upper and lower portion (superior and inferior portions), and used herein for purposes of defining relative direction and spatial orientation with respect to the wearer's body.

The term “coronal” is to be construed in the standard definition, referring to a vertical plane dividing the body into an anterior and posterior portions, or front and back, and used herein for purposes of defining relative direction and spatial orientation with respect to the wearer's body.

Headgear Support Structure Embodiments

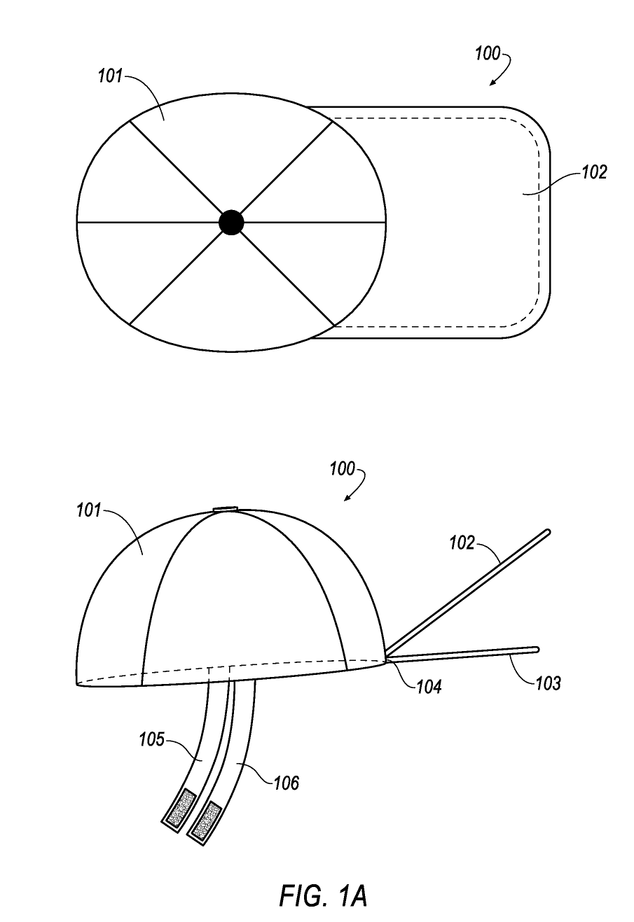

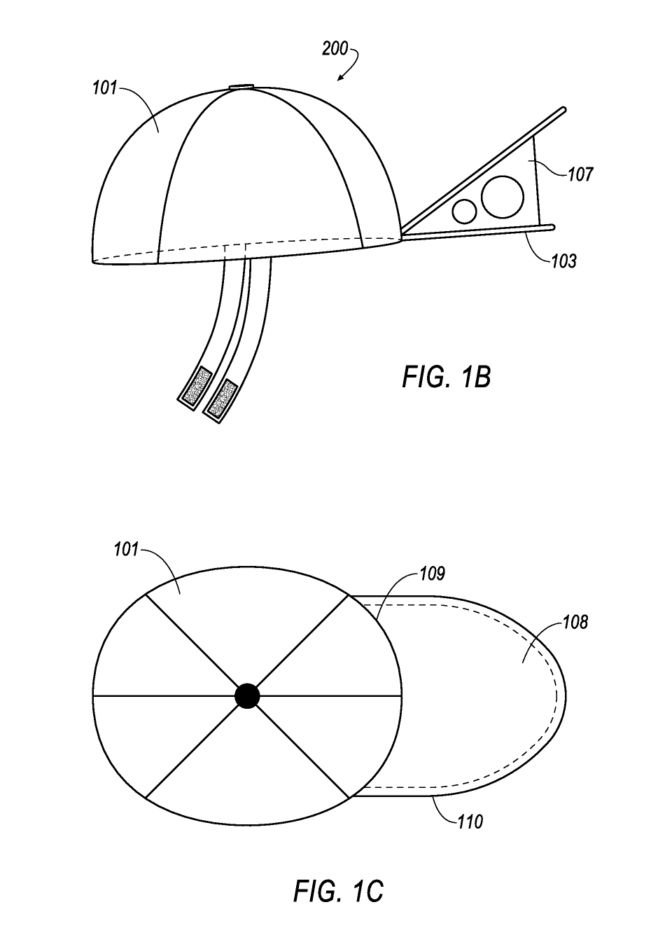

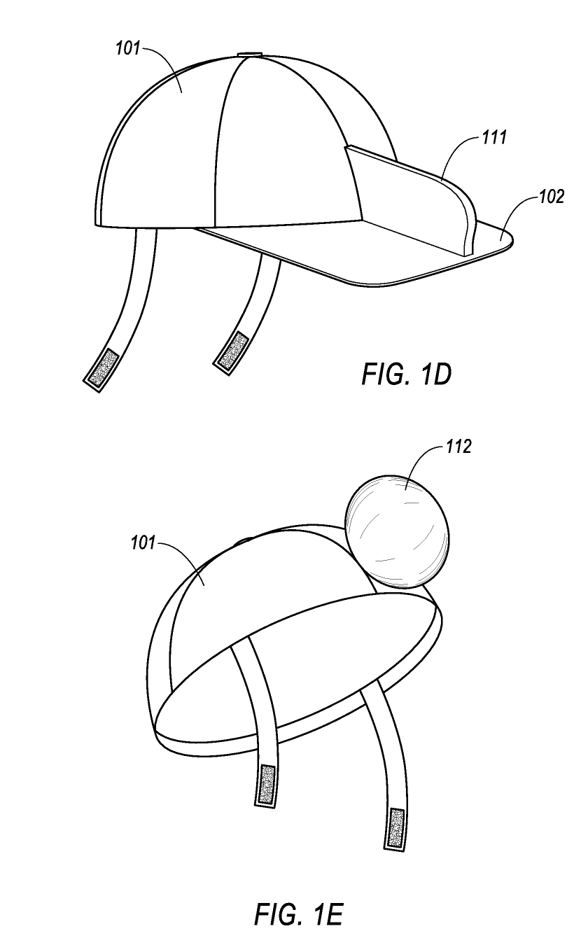

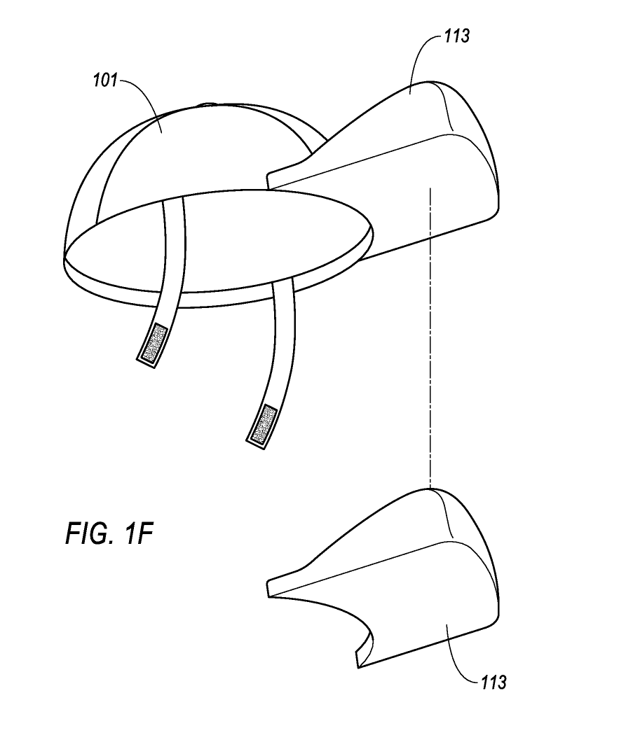

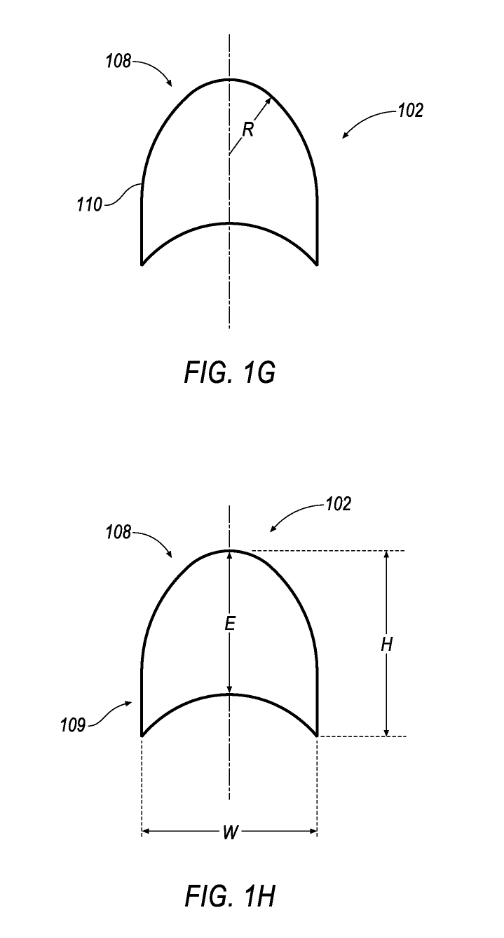

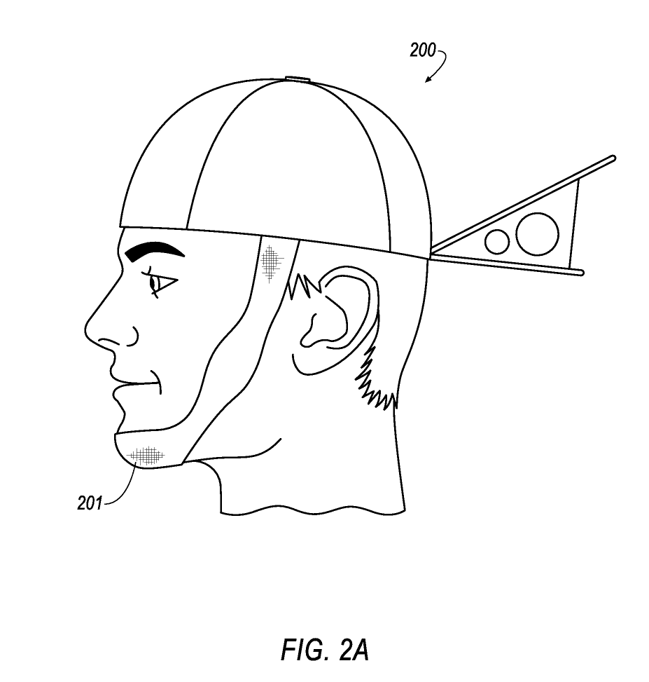

An example of the inventive headgear is given by the embodiment of the inventive headgear 100 is shown in FIG. 1a, as viewed from the top and side. In this example, the headgear support structure is a cap 101 to be worn on the cranium of the wearer in such a way that the cranium may be fully encircled by the headgear structure. In the example shown in FIG. 1a, chinstrap segments 105 and 106 extend from the rim of the cap. For all embodiments of the inventive headgear, the chinstrap may comprise two strap or band segments having one end affixed to the headgear structure, and fasteners disposed on the free ends of the strap segments. Examples of fasteners may include, but are not limited to: hook and loop (Velcro) patches, traditional belt-style buckles having a catch, cam and spring buckles, clip buckles, buttons, snaps, hooks, and the like.

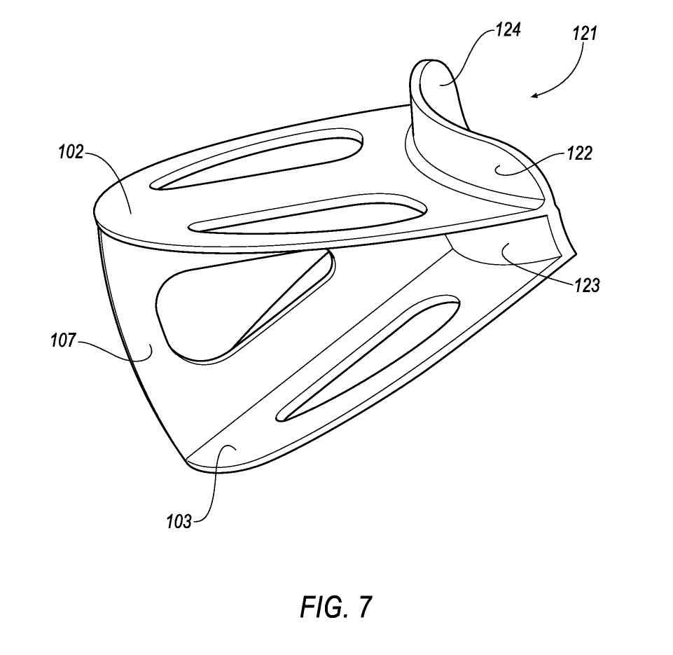

As is shown in the Example of FIG. 6, the cap 101 can include windows 119, such as nylon mesh windows, to enhance breathability.

Other embodiments of a headgear support structure may comprise more simple structures, such as, but not limited to, a headband encircling the cranium in the transverse plane, or a cranial band partially encircling the wearer's head in the coronal plane, similar to what is used in headphones, or a combination of the two. Additionally, the headgear structure may comprise a strap having two free ends, each free end having a fastening means, such as a hook-and-loop (Velcro) fastener patch, a buckle, a snap, a clip buckle, and the like.

Protrusion Embodiments

A protrusion assembly comprising two rigid bill-shaped structures 102 and 103 extends substantially rearward from the occipital (back or rear) portion of the cap. It will be understood that in other embodiments of the inventive headgear, the protrusions may assume different suitable shapes, such as those mentioned above in the definition of the term “bill-shaped structure,” without departing from the scope and spirit of the inventive concept.

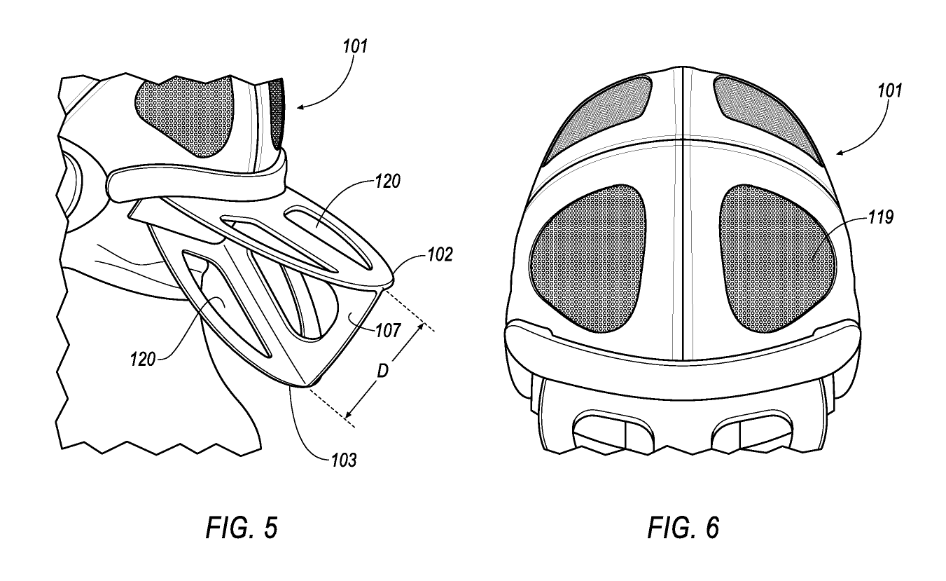

Referring to FIGS. 1a and 5, the two bill-shaped members 102 and 103 may be oriented horizontally or at small angles from the horizontal (with respect to the head held upright), and as depicted in the example of FIG. 1a, extending in the breadth and depth aspects, but having a small vertical extent. Furthermore, the two bill-shaped members 102 and 103 may both be affixed to the cap 100 close to its hem, or bottom border, forming a common joint 104 as seen in the side view of FIG. 1a. The particular shape of the bill-shaped members shown in the example of FIG. 1a is substantially rectilinear, but it will be understood that this form may equally be substituted by round shape embodiments as well, such as circular, ellipsoidal or more complex shapes, without departing from the scope of the invention. The bill-shaped members may also be curved or warped in the vertical dimension as well.

The two bill-shaped members 102 and 103 may also extend divergently rearward to form an angle between them. The angle serves to splay the two bill-shaped members in the sagittal plane, where members 102 and 103 diverge upwards and downwards, respectively, to provide a sufficient vertical aspect to the protrusion assembly that may impede folding or bending the protrusion assembly to an acute angle with respect to cap 101, which may reduce the effectiveness of the device to impede or block the wearer's head from assuming a supine position. Advantageously, this open architecture provides a light weight structure that accomplishes the goal of hindering head rotation during sleep, but does not cause the wearer's head to bear extra weight, which can be a source of discomfort to the wearer.

The splay angle between the two bill-shaped members may be maintained in a number of ways. One example is a rigid attachment joint formed by weld bonding each bill-shaped member separately to a rigid section of the headgear superstructure, such as a rigid plastic band sewn into the rim of a cap. Another example is a flat rigid structure folded into a V-shape, providing two rigid bill-shaped members splayed apart at a permanent angle, and integrally bonded to the headgear superstructure at the vertex of the V-shape, or by assembling a V-shaped where the V-shaped body may be fabricated from injection-molded plastic or by 3D printing of thermoset plastics or other types of polymers, and may furthermore be made as an integral solid piece with the headgear support structure, such as a headband or cap rim. The protrusion assembly may also be formed as an integral unit made from thermoset plastics or other polymers by injection molding or 3D printing techniques. The protrusion assembly may be sewn, riveted, bolted, glued, to the headgear superstructure.

Alternatively, the splay angle between the two members may be maintained by the method shown in FIG. 1b, depicting the placement of one or more rigid strut or web structures, such as exemplified by the fin-shaped web 107 in the angle formed between the two bill-shaped members 102 and 103 of the protrusion assembly. The one or more strut or web elements 107 maintain the separation between the two bill-shaped members 102 and 103, may be rigidly bonded to the two bill shaped members 102 and 103, or fabricated integrally as a single plastic or polymer-based unit by injection molding or 3D printing techniques. The strut or web elements 107 may take the form of simple rod struts or plate webs, such as the fin-shaped web shown in FIG. 1b. The addition of a strut or web element enables and reinforces the open architecture of the protrusion assembly, while at the same time maintaining the lightweight nature of the assembly.

Referring to FIG. 5, a distance D between splayed members 102, 103 can be, in one example, greater than approximately 0.5 inches. More particularly, the distance D may be between approximately 3 and 4 inches.

As is shown in FIG. 5, any of the members 102, 103 and the web 107 may include holes 120 which add to the open architecture of the protrusion assembly as discussed above.

In an alternative embodiment of the inventive headgear, as shown by example in FIG. 1c, each member 102 and 103 has a substantially ellipsoidal shape or a substantially parabolic shape, where the structures are tapered so that the distal ends 108 of the members converge to an apex, whereby the width of the distal ends is substantially narrow is comparison to the width of the proximal ends 109, providing a less stable pedestal for the wearer's head should the wearer succeed in rolling the head to a supine position.

As shown in FIG. 1c, the protrusion assembly is depicted to comprise members having tapered edges 110, giving the members a blunt nose at the apex. The apex or nose at the distal end 108 of the bill-shaped members may be blunt (rounded) or sharp (pointed). Should the wearer succeed in rolling the head into a supine position, the wearer's head may be forced to rest unstably upon the tapered or converging protrusion assembly, which may cause the head roll to the left or right, and not to remain in the supine position.

As is shown in FIG. 1g, in one example, the distal end 108 of the member 102 is rounded, and a radius R at the distal end 108 is less than approximately 3 inches. While member 102 is shown in FIG. 1g, it should be understood that preceding description applies to member 103 also.

The tapered or converging profile of the protrusion members may also increase the resistance to turning the head while wearing the inventive headgear, as the tapered or converging edge 110 provides a steeper and longer path upon which the head must roll in order to gain a supine position. Again, it is understood that the particular shape in FIG. 1e serves as an example of this embodiment, and that other basic tapered or converging shapes or profiles of the bill-shaped members may serve the same purpose, such as a triangular, trapezoidal, as well as parabolic or ellipsoidal, may be included in this embodiment. It may also be understood that more complex structures having edges that are tapered or converging to an apex at the distal end may be equivalents to the aforementioned embodiments.

In another alternative embodiment of the inventive headgear shown in the example depicted in FIG. 1d, the protrusion assembly may comprise a single bill-shaped member 102 having a vertically oriented rigid crest 111 affixed to it to prevent the bill-shaped member to being folded to an acute angle. The rigid crest may also be integral with the headgear 100 superstructure.

Referring now to FIG. 1h, a height H of the member 102 can be, in one example, between approximately 5 and 6 inches. The height H is the maximum distance between the distal end 108 and the proximal end 109.

A width W of the member 102 is defined at the proximal end 109. In one example, a ratio of the height H to the width W is greater than approximately 0.5. More particularly, the ratio is between approximately 1 and 1.25

An extension E of the member 102 is the minimum distance between the distal end 108 and the distal end 109 and corresponds to the distance from the wearer's head that the member 102 extends. The extension E, is, in one example, greater than about 2.5 inches and less than about 5 inches.

While member 102 is shown in FIG. 1h, it should be understood that preceding description applies to member 103 also.

In a further alternative embodiment of the inventive headgear, the protrusion may comprise a compact ball-shaped, or otherwise spheroid structure 112, as depicted in the example shown in FIG. 1e, as being affixed to the occipital portion of cap 101. The structure, which may have a substantially spherical, ellipsoidal or ovoid shape, may have principle dimensions, such as diameter, of sufficient size of at least 3 cm to impede or hinder the wearer's head from assuming a supine position. The protrusion may be fabricated from a rigid or semi-rigid material that exhibits some resiliency, such as a rubber material.

In the related embodiment of the inventive headgear shown in the example depicted in FIG. 1f, the protrusion assembly may comprise a single unit 113 having a substantial thickness in the vertical dimension, as well as having a sufficient horizontal aspect (breadth and depth), departing from the thin format of the above-described bill-shaped members. The thick protrusion embodiment may have a block-like character, or be wedge-shaped. It may be affixed to the headgear superstructure and extend rearward therefrom. The vertical dimension may be chosen to provide enough bulk behind the wearer's head to hinder the head from assuming a supine position, in the event that the protrusion is tilted substantially upward or downward by head movement during sleep, where the protrusion assembly may form an acute angle with respect to the occipital portion of the headgear superstructure, or the head. The single bulk unit may have mostly a rectilinear (box), rounded or wedge shape.

FIG. 1f also portrays an oblique view of the headgear, showing an example of a chinstrap comprising two strap portions or segments attached to the cap headgear structure, which may be fastened together when in use to secure the inventive headgear to the head. As a general description that may apply to all embodiments of the inventive headgear, the chinstrap segments are shown to have free ends upon which hook-and-loop (Velcro) fastener patches are disposed. The hook-and-loop fasteners may be integral with the strap portions, and may provide a means to adjust the tension of the chinstrap for wearer comfort and to optimize the functionality of the device. However, it will be understood that the strap segments may be endowed with other types of fasteners, such as buckles, clips, snaps, and the like.

Referring to FIG. 7, in one example, members 102, 103 and fin 107 extend from an end 121. The end 121 includes a lip 122. The lip 122 prevents the protrusion assembly from wobbling with respect to the wearer's head. The end 121 has a first side 123 from which the members 102, 103 and fin 107 protrude and a second side 124 opposite the first side 123. An attachment such as a hook-and-loop attachment can be provided on the second side 124 opposite the members 102, 103 and the fin 107 and/or along the lip 122 to attach the protrusion assembly to the cap 101. Attachments may also be provided on the first side 123 between the members 102, 103 on either side of the fin 107.

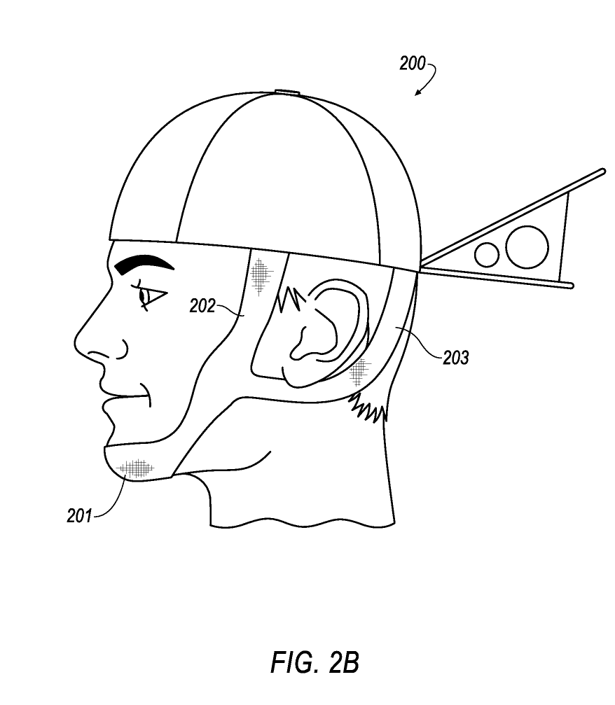

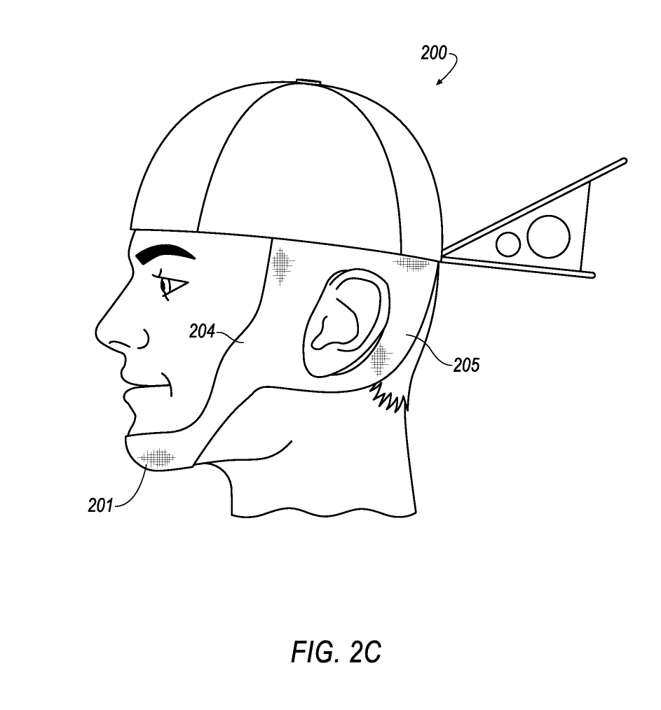

In FIG. 2a, the embodiment of the inventive headgear 200 is shown to further comprise a band or strap that serves as a chinstrap 201, extending downward from the cap headgear structure on both sides of the sagittal plane that divides the face along the cranial-caudal (vertical) axis, and passes under the wearer's chin serving to secure the cap to the wearer's head, and also to inhibit or otherwise restrict rotation of the cap during sleep. When worn in this manner, the chinstrap may partially encircle the wearer's head. In other embodiments, the chinstrap may fully encircle the wearer's head, passing over the wearer's cranium as well as under the chin, as an elastic or non-elastic band. A further function of the chinstrap may be to provide a tensile force on the lower jaw sufficient enough to hold the mouth closed during sleep.

FIG. 2b shows an alternative embodiment of the chinstrap, wherein the chinstrap 201 is bifurcated to attach to the headgear support structure cap 200, a first branch 202 passing in front, and a second branch 203 passing and directly behind the ear for more balanced distribution of tensile forces on the front and rear portions of the cap, as shown in the example, or other headgear support structure embodiments.

Similarly, the chinstrap may bifurcate into two wider branches 204 and 205 that run immediately in front and behind the ears, as shown in the example depicted in FIG. 2c. The larger width of the strap branches 204 and 205 allow the straps closer proximity to the ears compared to the branches 202 an 203 of FIG. 2b, even allowing contact with the ears, and serves to help prevent rotation of the inventive headgear by using the ears themselves as obstacles to rotation. In yet another alternative embodiment of the inventive headgear, two earflaps (not shown) may be used to accomplish the same purpose, whereby one earflap may be provided with a chinstrap and the other with receiving means for the chinstrap, or both earflaps may be provided with a portion of a chinstrap, and each earflap with a hole large enough to pass an ear through, so that the earflap may surround the ear when strapping the chinstrap under the chin. This embodiment is similar to, and has the same effect as the example depicted in FIG. 2c. It will also be understood that a pad or widened portion of the chinstrap that passes under the wearer's chin may be integral with the chinstrap structure.

In one example, the chinstrap is detachable from the headgear to provide a one-size-fits-all configuration.

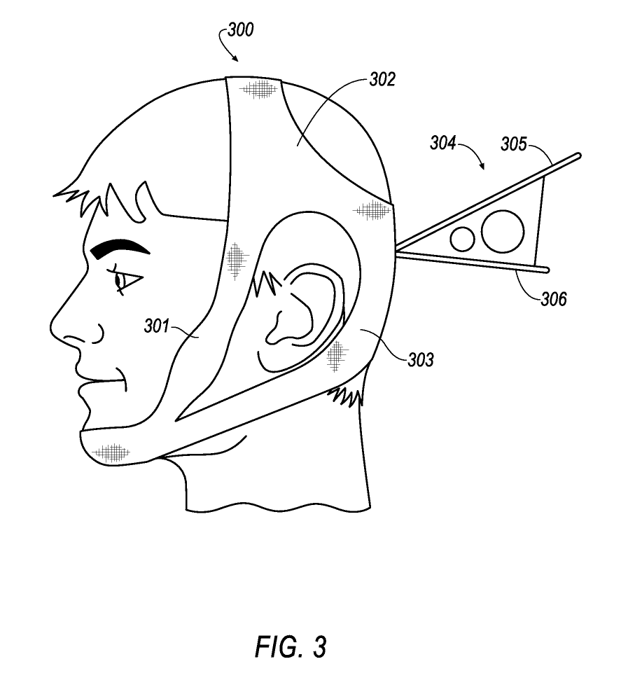

A further embodiment of the inventive headgear is shown in FIG. 3, where headgear superstructure 300 does not comprise a cap or similar structure completely covering the cranium of the user, but comprises a first band 301 encircling the wearer's head substantially along the cranial-caudal (vertical) axis, passing substantially under the chin and over the cranium of the wearer. The first band 301 may partially cover the wearer's cranium. The headgear superstructure 300 further comprises one or more bands 302 and 303 partially encircling the wearer's head substantially along the anterior-posterior (horizontal) axis of the wearer's head, extending rearward from and being integral with the first band, and partially covering the occipital portion of the wearer's head.

According to the embodiment example depicted In FIG. 3, one of the two lateral bands, the upper lateral band, extends from the upper portion of the first band, emanating from above the temple and extending substantially diagonally toward the occipital portion of the wearer's head. Also shown in FIG. 3 is the second of the two lateral bands, the lower lateral band, emanates from the chin diagonally upwards toward the occipital portion of the wearer's head, where it merges with the upper lateral band.

Referring again to FIG. 3, the bands 301-303 of the headgear superstructure 300 may be formed integrally by a molding process, a three-dimensional printing process, or by fastening the individual bands together by, but not limited to, stitching, welding or rivets, and may comprise at least partially elastic materials to provide tensile forces within the bands to secure the headgear superstructure to the wearer's head.

Alternatively, the headgear superstructure may be formed from substantially non-elastic materials, as well as comprise re-usable fastening means such as hook-and-loop (Velcro) fasteners, snaps, buttons, zippers, belt buckles, buckle clips, and the like to secure the headgear superstructure to the wearer's head.

Still referring to FIG. 3, a protrusion assembly 304 extends rearward from the occipital portion of the headgear superstructure 300. The protrusion assembly 304 depicted in FIG. 3 is an example of one of several possible protrusions that may be employed. Thus, the protrusion assembly may comprise the two bill-shaped members 305 and 306 divergently extending from the occipital portion of the headgear superstructure as described above, or may comprise other examples without departing from the scope and spirit of this disclosure.

### EXAMPLE OF USE

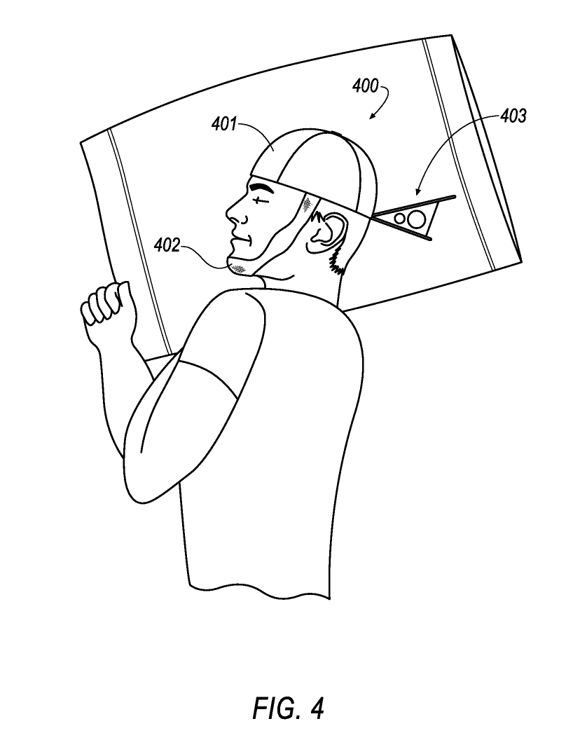

An example deployment of the headgear is shown in FIG. 4, which depicts a subject wearing the preferred embodiment of the inventive headgear 400 and is induced to remain in the lateral incumbent sleeping position by the bill-shaped protrusions. The cap 401 is held securely on the subject's head by chinstrap 402, which also holds the mouth closed, forcing the subject to breathe primarily through the nose while the head rests in a lateral position. The inventive headgear 400 includes a protrusion assembly 403.

The embodiments of the inventive headgear described herein serve as examples for the purposes of this disclosure, and are by no means meant to be construed as limiting the invention to those specified. It is to be understood that many equivalent permutations of the herein described embodiments may be conceived without departing from the scope and spirit of the invention.

Although an embodiment of this invention has been disclosed, a worker of ordinary skill in this art would recognize that certain modifications would come within the scope of this disclosure. For that reason, the following claims should be studied to determine the true scope and content of this disclosure.

1. A head-wearable device for hindering the assumption of a supine position by the wearer's the head during sleep, comprising:
(a) A headgear support structure for wearing on the head and having a shape that at least partially encircles the cranium of the wearer;
(b) A band structure for securing the headgear support structure to the wearer's head, wherein the band structure encircles at least a portion of the wearer's head substantially along the cranial-caudal axis of the wearer's head, and passing under the wearer's chin for securing and holding the headgear support structure to the head of the wearer, said band structure being affixed to the headgear support structure on both sides of the sagittal plane dividing the wearer's head, such that the combination of the headgear support structure and the band structure forms a headgear superstructure; and
(c) A protrusion assembly affixed to the headgear superstructure for hindering the wearer's head from assuming a supine position during sleep, said protrusion assembly extending rearwardly from the headgear superstructure into the space substantially behind the occipital portion of the wearer's head, where the protrusion assembly comprises first and second members separated by a fin, wherein a distance between the first and second members at a distal end of each of the first and second members is greater than about 0.5 inches.

2. The head-wearable device of claim 1, wherein the head support structure covers at least a portion of the cranium of the wearer.

3. The head-wearable device of claim 2, wherein the head support structure is a cap.

4. The head-wearable device of claim 3, wherein the cap includes at least one window.

5. The head-wearable device of claim 4, wherein the at least one window comprises nylon mesh.

6. The head-wearable device of claim 1, the fin includes a strut.

7. The head-wearable device of claim 1, wherein fin is a web.

8. The head-wearable device of claim 1, wherein the first and second members are bill-shaped and have a blunt vertex.

9. The head-wearable device of claim 8, wherein a radius of the first and second members at the blunt vertex is less than approximately 3 inches.

10. The head-wearable device of claim 1, wherein a height of the first and second members is between approximately 5 and 6 inches.

11. The head-wearable device of claim 1, wherein each of the first and second members has a height and a width, and a ratio of the height to the width of each of the first and second members is greater than approximately 0.5

[symbol]

12. The head-wearable device of claim 11, wherein the ratio is between approximately 1 and 1.25.

13. The head-wearable device of claim 1, wherein the protrusion assembly extends greater than approximately 2.5 inches from the head of the wearer.

14. The head-wearable device of claim 1, wherein the distance between the first and second members at a distal end of each of the first and second members is between approximately 3 and 4 inches.

15. A method of sleeping comprising the steps of:
(a) wearing a head-wearable device for hindering the assumption of a supine position by the wearer's head during sleep, comprising a headgear support structure for wearing on the head and having a shape that at least partially encircles the cranium of the wearer, the a head-wearable device including:
a band structure for securing the headgear support structure to the wearer's head, wherein the band structure encircles at least a portion of the wearer's head substantially along the cranial-caudal axis of the wearer's head, and passing under the wearer's chin for securing and holding the headgear support structure to the head of the wearer, said band structure being affixed to the headgear support structure on both sides of the sagittal plane dividing the wearer's head, such that the combination of the headgear support structure and the band structure forms a headgear superstructure, and said band structure being constructed to hold a user's mouth closed when worn; and
a protrusion assembly affixed to the headgear superstructure for hindering the wearer's head from assuming a supine position during sleep, said protrusion assembly extending rearwardly from the headgear superstructure into the space substantially behind the occipital portion of the wearer's head, where the protrusion assembly comprises first and second members separated by a fin; and
(b) sleeping, the head-wearable device preventing the wearer's head from rotating from a substantially lateral orientation to a substantially supine orientation.

16. The method of claim 15, wherein the first and second members are bill-shaped and have a blunt vertex.

17. The method of claim 14, wherein a distance between the first and second members at a distal end of each of the first and second members is greater than about 0.5 inches

[symbol]

18. The method of claim 15, wherein the protrusion assembly extends greater than approximately 2.5 inches from the head of the wearer.

19. The method of claim 15, further comprising the step of adjusting the band structure to the wearer's head.

20. The method of claim 19, further wherein the adjusting step is accomplished via hook-and-loop fasteners.
